(19) United States
(12) Patent Application Publication
(10) Pub. No.: US 2008/0299294 A1
(43) Pub. Date: Dec. 4, 2008

(54) ANTHRACENE COMPOUND AND ORGANIC LIGHT EMITTING DEVICE USING THE SAME

(75) Inventor: Feng-Wen Yen, Hsin-Chu (TW)

Correspondence Address: FENG-WEN YEN, 2F, NO. 21, R&D ROAD II, SCIENCE-BASED INDUS. PARK, HSIN-CHU, TW

(73) Assignee: Feng Wen Yen, Hsin-Chu (TW)

(21) Appl. No.: 11/757,984

(22) Filed: Jun. 4, 2007

(51) Int. Cl. C09K 11/06; C07C 15/28; C07C 25/22

(52) U.S. Cl. 427/66; 570/129; 570/183; 585/26

(57) ABSTRACT

The present invention discloses an anthracene compound which can be used as emitting material in organic electroluminescence devices is disclosed. The mentioned anthracene compound is represented by the following formula [I]

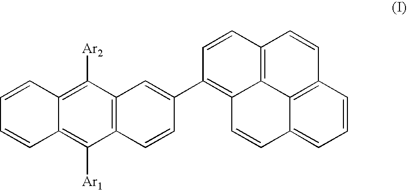

Wherein Ar₁ and Ar₂ are the same or different and each is selected from a substituted or unsubstituted phenyl group, a substituted or unsubstituted naphthyl group, a substituted or unsubstituted biphenyl group, a substituted or unsubstituted fluorenyl group, a substituted or unsubstituted anthryl group.

### BACKGROUND OF THE INVENTION

1. Field of the Invention

This invention is related to an anthracene compound and an organic light emitting device using the anthracene compound. More specifically, this invention relates to an anthracene compound having chemical structure represented as the following general formula(I) and an organic light emitting using the anthracene compound for a emitting layer.

2. Description of the Prior Art

Anthracene compound used as emitting layer for organic light emitting device was firstly described in “Thin Solid Films, 94, 171(1982)”, J. shi and C. W. Tang described 9,10-di(2-naphthyl)anthracene(AND) derivatives as emitting host in “APPL. Phys. Lett., 80,3201(2002)”, EP 1,156,536(2001) described anthracene derivatives 2-(t-butyl)-9,10-di-(2-naphthyl) anthracene (TBADN) as a blue host. U.S. Pat. No. 6,465,115 B2 described Anthracene derivatives using for hole transporting layer, Synth. Met., 141,245(2004) described anthracene derivatives 2,3,6,7-tetra-methyl-9-10-dinaphthyl-anthracene(TMADN) as a blue host material, Chin H. Chen et al, “Efficient Blue Organic Electroluminescent Devices Based on a Stable Blue Host material”, SID 2004 digest, described anthracene derivatives 2-methyl-9,10-di(2-naphthyl)anthracene (MADN) as a blue host material, US 2005/0064233A1 described bis-anthracene derivatives as emitting host. But these anthracene derivatives are still needed corresponding to increasing thermal stability and practical operation durability. Especially the half-lifetime and efficiency needed to be improved.

### SUMMARY OF THE INVENTION

In accordance with the present invention, anthracene compounds and their use for emitting layer of organic light emitting device are provided. These anthracene compounds can overcome the drawbacks of the mentioned conventional materials. In order to obtain better thermal stability, we introduced pyrene group into 2-position moiety of anthracene compound, so as to enlargement of molecular size, enhances thermal degradation temperature (T_{d}) and the stability of the amorphous glassy state and increasing its charge carrier mobility.

One object of the present invention is to improve the Tg and heat-resistant physical characteristic of anthracene compound.

Another object of the present invention is to applied these anthracene compounds for light emitting layer of organic light emitting devices and improve the half-lifetime, lower driving voltage, lower power consumption and increasing the efficiency.

This present invention does have the economic advantages for industrial practice. Accordingly, the present invention discloses a anthracene compound which can be used for organic light emitting device is disclosed. The mentioned anthracene compound is represented by the following formula(I)

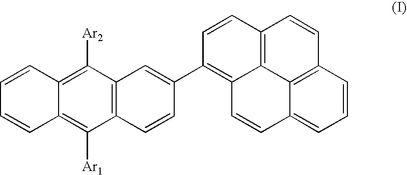

Wherein Ar₁ and Ar₂ are the same or different and each is selected from a substituted or unsubstituted phenyl group, a substituted or unsubstituted naphthyl group, a substituted or unsubstituted biphenyl group, a substituted or unsubstituted fluorenyl group, a substituted or unsubstituted anthryl group.

### BRIEF DESCRIPTION OF THE DRAWINGS

FIG. 1 show an example of organic light emitting device in the present invention 1 is transparent electrode, 5 is metal electrode, 2 is hole transporting layer which is deposited onto 1, 3 is green emitting layer which is deposited onto 2, 4 is electron transporting layer which is deposited onto 3.

FIG. 2 show an example of organic light emitting device in the present invention. 1 is transparent electrode, 5 is metal electrode, 2 is hole transporting layer which is deposited onto 1, 6 is blue emitting layer which is deposited onto 2, 4 is electron transporting layer which is deposited onto 3.

### DESCRIPTION OF THE PREFERRED EMBODIMENTS

What probed into the invention is anthracence compound and organic light emitting device using the compound. Detailed descriptions of the production, structure and elements will be provided in the following to make the invention thoroughly understood. Obviously, the application of the invention is not confined to specific details familiar to those who are skilled in the art. On the other hand, the common elements and procedures that are known to everyone are not described in details to avoid unnecessary limits of the invention. Some preferred embodiments of the present invention will now de described in greater detail in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

### Definition

The term “thermal degradation temperature (T_{d})” herein refers to the temperature when the weight loss of a heated specimen being 0.5 wt %. “T_{g}” herein refers to the glass transition temperature and “Tₘ” herein refers to melting point.

In a first embodiment of the present invention, anthracene compound which can be used as emitting layer of OLEDs is disclosed. The mentioned anthracene compound is represented by following formula(I)

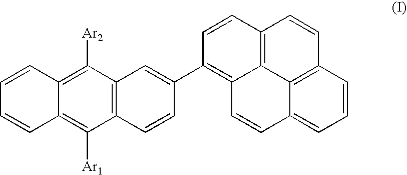

Wherein Ar₁ and Ar₂ are the same or different and each is selected from a substituted or unsubstituted phenyl group, a substituted or unsubstituted naphthyl group, a substituted or unsubstituted biphenyl group, a substituted or unsubstituted fluorenyl group, a substituted or unsubstituted anthryl group.

In this embodiment, some anthracene compounds of the present invention will be shown, but anthracene compound is not limited to the following examples.

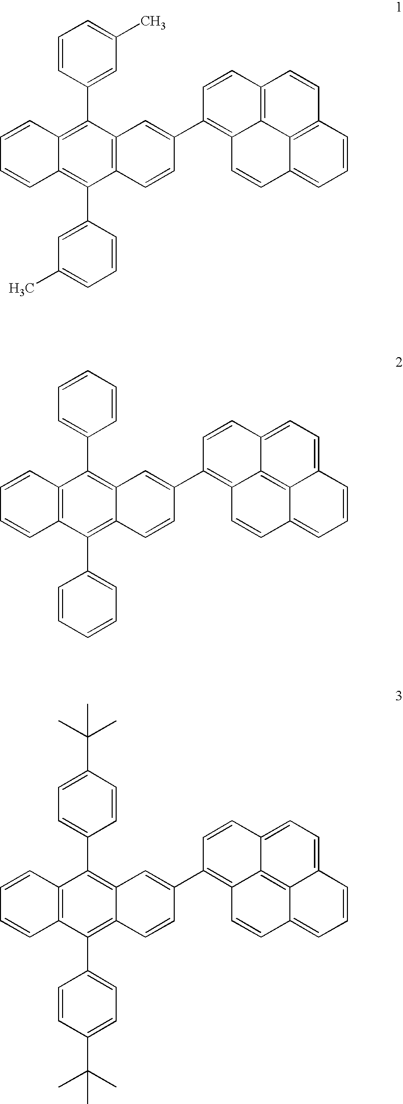

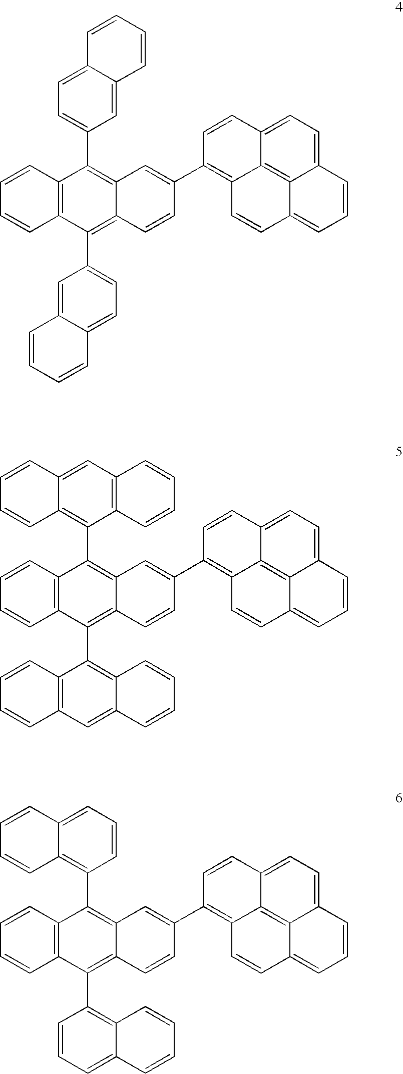

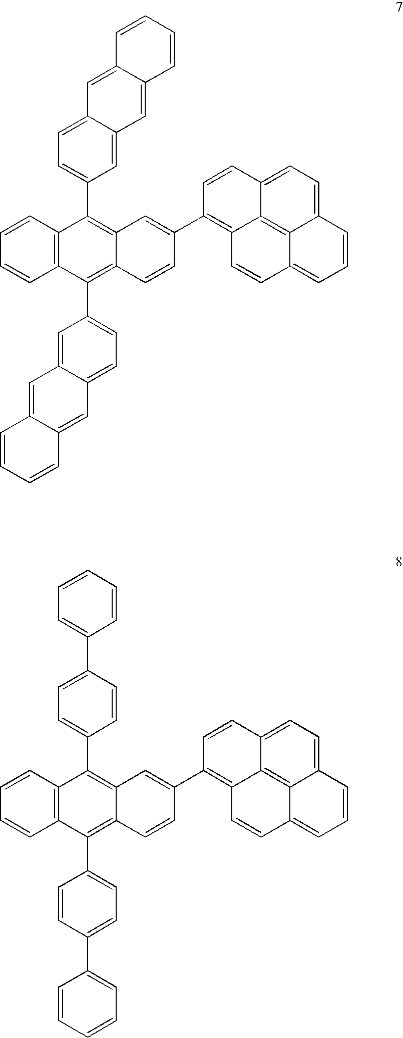

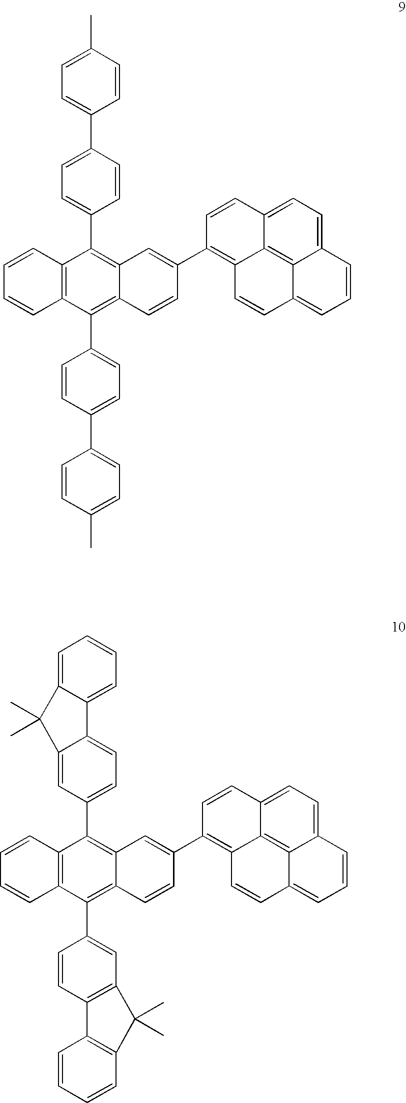

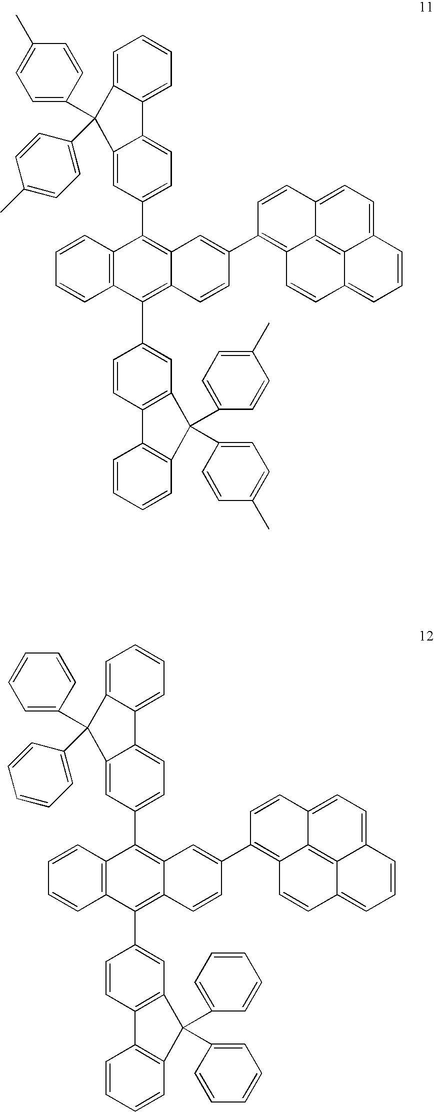

The present invention will be described more specifically based on the following examples.

### EXAMPLE 1

### Synthesis of Example Compound No.2

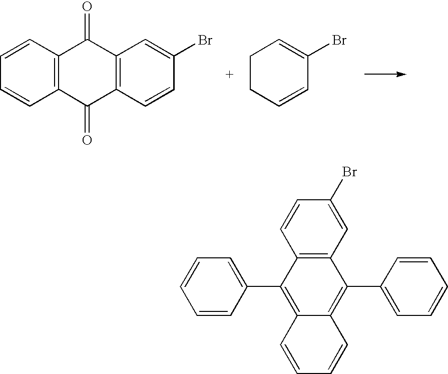

Synthesis of 2-Bromo-9,10-diphenylanthracene

25 g (159 mmole) of bromobenzene was placed in an 500 ml round bottom flask, and was dissolved in dry THF. The solution was cooled down to −70° C. by dry ice/acetone, then 1.6M n-Butyl lithium 67 ml was added slowly over 30 minutes. The reaction was stirred for 2 hours while the reaction temperature was maintained at −70° C., 10 g (34.8 mmole) of 2-bromoanthraquinone (manufactured by Tokyo Chemical Industry Co., LTD.,) was added to the cooled reaction mixture, the reaction was stirred for overnight at room temperature. Then, 120 ml of 10% HCl_{(aq)} was added to the resulting solution, and stirred for 30 minutes, the organic layer was separated, and was evaporated to dryness, the brown color residue was dissolved in 60 ml of acetic acid, and 29 g of SnCl₂. 2H₂O were added to the brown solution. The reaction mixture was heated to reflux for 6 hours, and then, cool down to room temperature. Thus, The brown product was collect be filtration, (6 g, 42.6%)

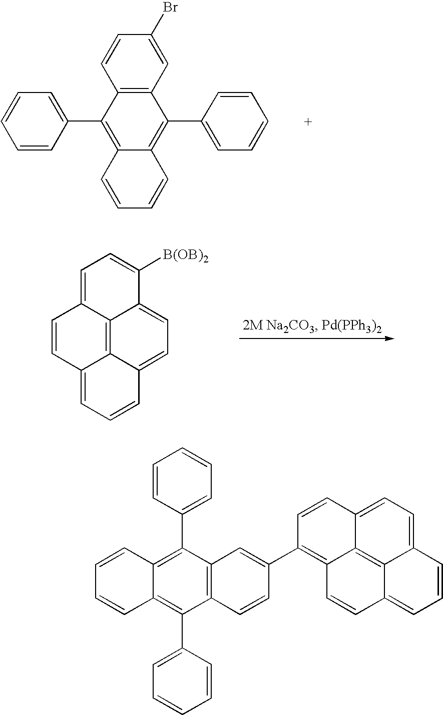

Synthesis of 2-pyrenyl-9,10-diphenylanthracene

2-bromo-9,10-diphenylanthracene (5 g 12 mmole) was placed into a 250 ml round bottom flask along with pyrene-1-boronic acid (4.5 g 18 mmole), THF 100 ml, and 2.0M aqueous sodium carbonate (18 ml). The reaction mixture was purged with nitrogen for 30 minutes and then Pd(PPh₃)₄(0.42 g) was added. The reaction was heated at reflux for 18 hours and then cooled down to room temperature. A yellow-green product was collected by filtration. The solid was wash with MeOH/H₂O and dry in vacuum to get crude product. Further purification was achieved by sublimation (4.4 g, 67.7%). The product was identified through EI-MS measurement, m/s=530

### EXAMPLE 2

### Synthesis of Example Compound No. 4

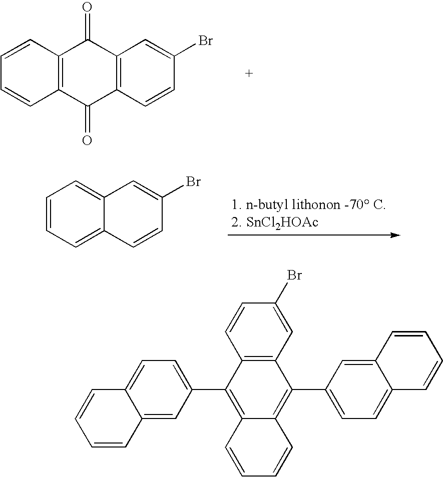

Synthesis of 2-Bromo-9,10-di(naphthalen-2-yl)anthracene

4.3 g (21 mmole) of 2-bromonaphthalene was placed in an 100 ml round bottom flask, and was dissolved in dry THF. The solution was cooled down to 31 70° C. by dry-ice/acetone, then 1.6M n-Butyllithium 13 ml (21 mmole) was added slowly over 30 minutes. The reaction was stirred for 2 hours while the reaction temperature was maintained at −70° C., 2-bromoanthraquinone 2 g (6.9 mmole; manufactured by Tokyo Chemical Industry Co., LTD.,) was added to the cooled reaction mixture, the reaction was stirred for overnight at room temperature. Then, 30 ml of 10% HCl was added to the resulting solution, and stirred for 30 minutes, the organic layer was separated, and was evaporated to dryness, the brown color residue was dissolved in 50 ml of acetic acid, and 1.6 g of SnCl₂. 2H₂O were added to the brown solution. The reaction mixture was heated to reflux for 6 hours, and then, cooled down to room temperature. Thus, the Brown product was collect by filtration. (2.47 g, 70.4%)

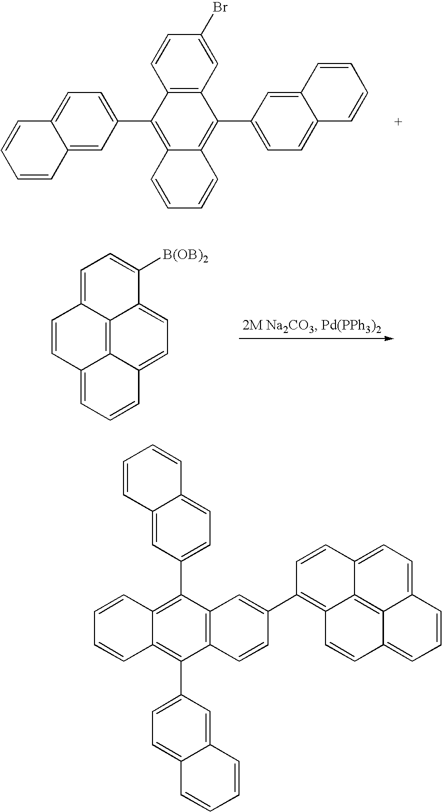

Synthesis of 2-pyrenyl-9.10-di(naphthalen-2-yl)anthracene

2-bromo-9,10-di(naphthalen-2-yl)anthracene (2.5 g 4.9 mmole) was placed into a 100 ml round bottom flask along with pyrene-1-boronic acid (1.33 g, 5.4 mmole), toluene (30 ml), EtOH 910 ml), and 2.0M aqueous sodium carbonate (9.8 ml). The reaction mixture was purged with nitrogen for 30 minutes and then Pd(PPh₃)₄(0.01 g), 2-dicyclohexylphosphinobiphenyl (0.01 g) was added. The reaction was heated at reflux for 18 hours and then cooled down to room temperature. A yellow-green product was collected by filtration. The solid was wash with MeOH/H₂O and dry in vacuum to get crude product. Further purification was achieved by sublimation (2.0 g, 64.5%). The product was identified through EI-MS measurement, m/s=631

### EXAMPLE 3

### Synthesis of Example Compound No.6

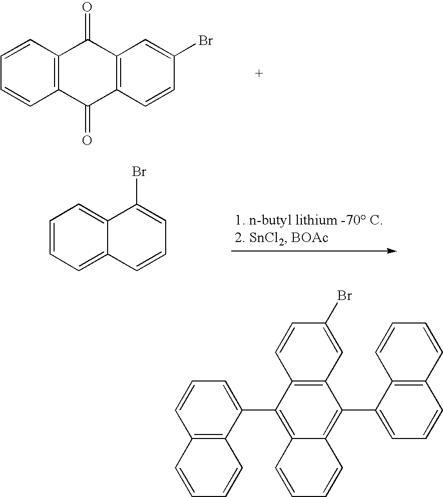

Synthesis of 1-Bromo-9,10-di(naphthalen-1-yl)anthracene

4.3 g (21 mmole) of 1-bromonaphthalene was placed in an 100 ml round bottom flask, and was dissolved in dry THF. The solution was cooled down to −70° C. by dry-ice/acetone, then 1.6M n-Butyllithium 13 ml (21 mmole) was added slowly over 30 minutes. The reaction was stirred for 2 hours while the reaction temperature was maintained at −70° C., 2-bromoanthraquinone 2 g (6.9 mmole; manufactured by Tokyo Chemical Industry Co., LTD.,) was added to the cooled reaction mixture, the reaction was stirred for overnight at room temperature. Then, 30 ml of 10% HCl was added to the resulting solution, and stirred for 30 minutes, the organic layer was separated, and was evaporated to dryness, the brown color residue was dissolved in 50 ml of acetic acid, and 1.6 g of SnCl₂. 2H₂O were added to the brown solution. The reaction mixture was heated to reflux for 6 hours, and then, cooled down to room temperature. Thus, The Brown product was collect by filtration (2.22 g, 63.1%)

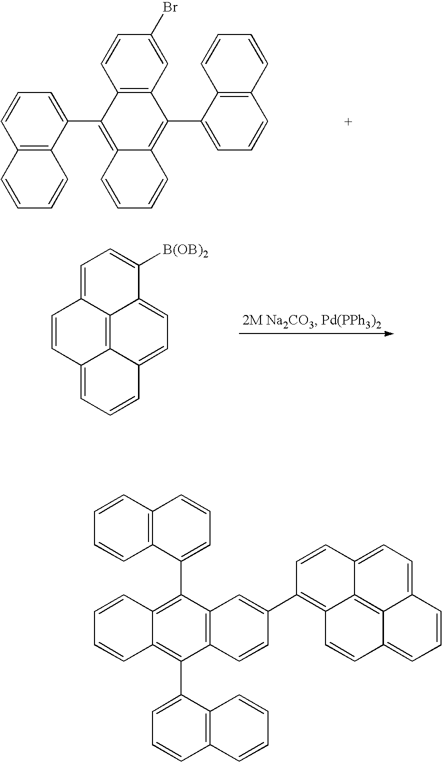

Synthesis of 2-pyrenyl-9,10-di(naphthalen-1-yl)anthracene

2-bromo-9,10-di(naphthalen-1-yl)anthracene (2.2 g 4.3 mmole) was placed into a 100 ml round bottom flask along with pyrene-1-boronic acid (1.16 g 4.73 mmole), toluene (30 ml), EtOH (10 ml), and 2.0M aqueous sodium carbonate (8.6 ml). The reaction mixture was purged with nitrogen for 30 minutes and then Pd(PPh₃)₄ (0.01 g), 2-dicyclohexylphosphinobiphenyl (0.01 g) was added. The reaction was heated at reflux for 18 hours and then cooled down to room temperature. A yellow-green product was collected by filtration. The solid was wash with MeOH/H₂O and dry in vacuum to get crude product. Further purification was achieved by sublimation (1.45 g, 53.5%). The product was identified through FAB-MS measurement, m/s=630

### EXAMPLE 4

### Synthesis of Example Compound No.10

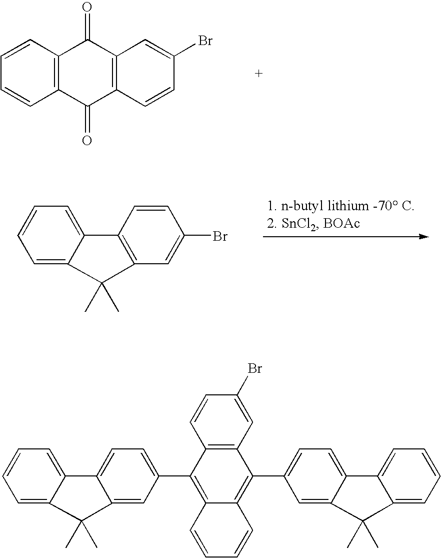

Synthesis of 2-Bromo-9.10-di(9,9-dimethylfluorene-2-yl) anthracene

16.12 g (59 mmole) of 2-bromo-9,9-dimethylfluorene was placed in an 250 ml round bottom flask, and was dissolved in dry THF. The solution was cooled down to −70° C. by dry-ice/acetone, then 1.6M n-Butyllithium 36.9 ml (59 mmole) was added slowly over 30 minutes. The reaction was stirred for 2 hours while the reaction temperature was maintained at −70° C., 2-bromoanthraquinone 2.8 g (9.8 mmole; manufactured by Tokyo Chemical Industry Co., LTD.,) was added to the cooled reaction mixture, the reaction was stirred for overnight at room temperature. Then 30 ml of 10% HCl was added to the resulting solution, and stirred for 30 minutes, the organic layer was separated, and was evaporated to dryness, the brown color residue was dissolved in 60 ml of acetic acid, and 2 g of SnCl₂. 2H₂O were added to the brown solution. The reaction mixture was heated to reflux for 6 hours, and then, cooled down to room temperature. Thus, The Brown product was collect by filtration (2.8 g, 50.2%)

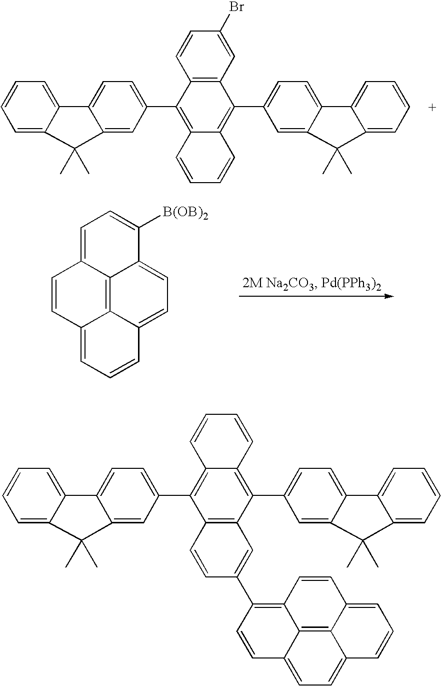

Synthesis of 2-pyrenyl-9.10-di(9,9-dimethylfluoren-2-yl)anthracene

2-bromo-9,10-di(9.9-dimethylfluoren-2-yl)anthracene (1.68 g 2.6 mmole) was placed into a 100 ml round bottom flask along with pyrene-1-boronic acid (0.8 g 3.3 mmole), toluene (30 ml), EtOH (10 ml), and 2.0M aqueous sodium carbonate (5 ml). The reaction mixture was purged with nitrogen for 30 minutes and then Pd(PPh₃)₄(0.02 g), 2-dicyclohexylphosphinobiphenyl (0.01 g) was added. The reaction was heated at reflux for 18 hours and then cool down to room temperature. A yellow-green product was collected by filtration. The solid was wash with MeOH/H₂O and dry in vacuum to get crude product. Further purification was achieved by sublimation (0.96 g, 48.5%). The product was identified through FAB-MS measurement, m/s=762

### General Method of Producing Oleds

ITO-coated glasses with 15 Ωm⁻¹ and 1500 μm in thickness are provided (purchased from Sanyo vacuum, hereinafter ITO substrate) and cleaned in a number of cleaning steps in an ultrasonic bath (e.g. detergent, deionized water). Before vapor deposition of the organic layers, cleaned ITO substrates are further treated by UV and ozone.

These organic layers are applied onto the ITO substrate in order by vapor deposition in a high-vacuum unit (10⁻⁶ Torr), such as: resistively heated quartz boats. The thickness of the respective layer and the vapor deposition rate (0.1˜0.3 nm/sec) are precisely monitored or set with the aid of a quartz-crystal monitor. It is also possible, as described above, for individual layers to consist of more than one compound, i.e. in general a host material doped with a guest material. This is achieved by co-vaporization from two or more sources.

N,N′-Bis(naphthalene-1-yl)-N,N′-bis(phenyl)-benzidine (NPB) is most widely used as the hole transporting layer and Tris-(8-hydroxyquinoline) aluminum (Alq₃) is most widely used as the electron transporting/light emitting layer in OLEDs for its high thermal stability and good film forming property. It is reported that the thermal degradation temperature (T_{d}) of Alq₃ is about 303° C. 2,3,6,7-Tetrahydro-1,1.,7-tetramethyl-1H, 5H, 11H-10-(2-benzo-thiazolyl) quinolizi no-[9,9a, 1 gh] coumarin (C545T) is widely used as the green quest to co-vaporization with host (Alq₃) for green emissive layer. 2,5,8,11-tetra-tert-butylperylene(TBPe) is widely used as the blue guest to co-vaporization with host (9,10-di(2-naphthyl)anthracene(AND) or 2-(t-butyl)-9,10-di-(2-naphthyl)anthracene (TBADN)) for blue emissive layer.

A typical OLED consists of low work function metals, such as Al, Mg, Ca, Li and K, as the cathode by thermal evaporation, and the low work function metals can help electrons injecting the electron transporting layer from cathode. In addition, for reducing the electron injection barrier and improving the OLED performance, a thin-film electron injecting layer is introduced between the cathode and the electron transporting layer. Conventional materials of electron injecting layer are metal halide or metal oxide with low work function, such as: LiF, MgO, or Li₂O.

On the other hand, after the OLEDs are fabricated, EL spectra and CIE coordination are measured by using a PR650 spectra scan spectrometer. Furthermore, the current/voltage, luminescence/voltage and yield/voltage characteristics are taken with a Keithley 2400 programmable voltage-current source. The above-mentioned apparatuses are operated at room temperature (about 20° C.) and under atmospheric pressure.

### EXAMPLE 5

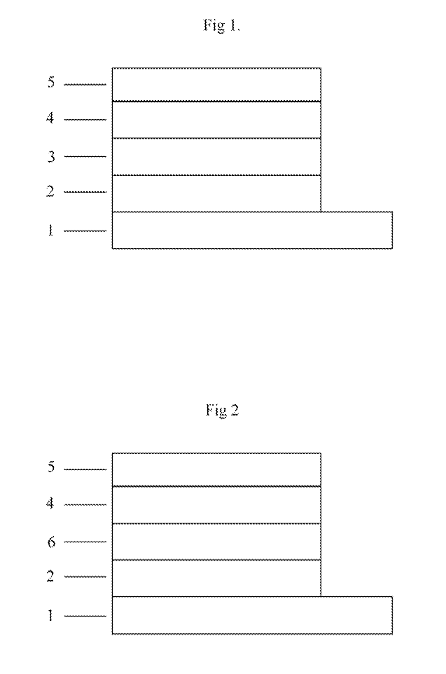

Using a procedure analogous to the abovementioned general method, green and blue OLEDs having the following device structure were produced:
Green OLEDs: ITO/NPB (600 Å)/Emitting host doped 2% C545T (300 Å)/Alq₃ (250 Å)/LiF (5 Å)/Al (1200 Å), the crossed-sectional views as FIG. 1
Blue OLEDs: ITO/NPB (600 Å)/Emitting host doped 1% TBPe (300 Å)/Alq₃ (150 Å)/LiF (5 Å)/Al (1200 Å), the crossed-sectional views as FIG. 2

The luminance, Yield and half-life time of green devices testing report as Table 1 and blue devices testing report as Table 2.

TABLE 1

| | | | Half-lifetime(hour) |
|---|---|---|---|
| | Luminance | Yield | Initial luminance = |
| Emitting host | (cd/m²) | (cd/A) | 3000 (cd/m²) |
| Compound No. 2 | 16900 | 11.3 | 85 |
| Compound No. 4 | 58100 | 11.7 | 106 |
| Compound No. 6 | 46300 | 9.0 | 148 |
| Compound No. 10 | 53300 | 12.8 | 175 |
| Alq₃ | 26400 | 11.5 | 63 |

Applied Voltage = 9 V

TABLE 2

| | | | Half-lifetime(hour) |
|---|---|---|---|
| | Luminance | Yield | Initial luminance = |
| Emitting host | (cd/m²) | (cd/A) | 3000 (cd/m²) |
| Compound No. 2 | 9600 | 1.83 | 75 |
| Compound No. 4 | 11600 | 1.96 | 88 |
| Compound No. 6 | 12900 | 1.58 | 86 |
| Compound No. 10 | 10200 | 2.08 | 96 |
| ADN | 6940 | 1.96 | 18 |
| TBADN | 7220 | 2.09 | 12 |

Applied Voltage = 9 V

### EXAMPLE 6

The physical characteristics of anthracene compounds are comparable with Alq₃, ADN, TBADN. The data are shown in Table 3

TABLE 3

| | Mp(° C.) | Tg(° C.) | TGA(° C.) | PL(nm) |
|---|---|---|---|---|
| Compound No. 2 | 145 | 135 | 365 | 452 |
| Compound No. 4 | 383 | 159 | 430 | 456 |
| Compound No. 6 | 361 | 141 | 416 | 451 |
| Compound No. 10 | — | — | 437 | 456 |
| Alq₃ | 417 | — | 300 | 512 |
| ADN | 386 | 155 | 335 | 425 |
| TBADN | 291 | 138 | 327 | 431 |

In the above preferred embodiments (Table 1-Table 2), we show that the anthracene compounds of this invention have efficient emitting layer properties than comparable Alq_{3,} ADN, TBADN with higher half-life time and practical operation durability. High luminance than Alq_{3,} ADN, TBADN has also been achieved at a driving voltage of 9 V using the mentioned anthracene compounds for green-emitting and blue-emitting of organic electroluminescent devices.

In Table 3, we show that anthracene compounds of this invention have higher heat-resistant physical characteristic (TGA) and Tg comparable with Alq₃, ADN, TBADN.

To sum up, the present invention discloses anthracene compound which can be used as emitting layer of OLEDs in organic electroluminescence devices is disclosed. The mentioned anthracene compound is represented by the following formula (I)

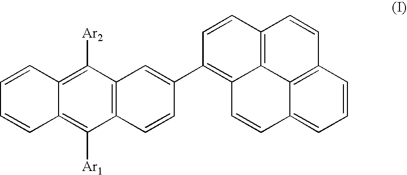

Wherein Ar₁ and Ar₂ are the same or different and each is selected from a substituted or unsubstituted phenyl group, a substituted or unsubstituted naphthyl group, a substituted or unsubstituted biphenyl group, a substituted or unsubstituted fluorenyl group, a substituted or unsubstituted anthryl group.

Obvious many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. An anthracene compound with a general formula (I) as following: 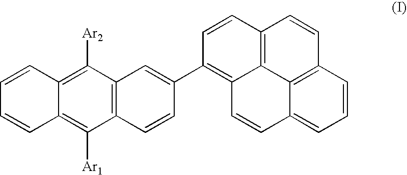
Wherein Ar₁ and Ar₂ are the same or different and each is selected from a substituted or unsubstituted phenyl group, a substituted or unsubstituted naphthyl group, a substituted or unsubstituted biphenyl group, a substituted or unsubstituted fluorenyl group, a substituted or unsubstituted anthryl group.

2. The compound as claimed in claim 1, wherein the naphthyl group is represented by the general formula (II): 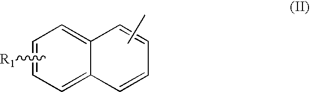
wherein R₁ is selected from a hydrogen atom, a halogen atom, a substituted or unsubstituted amino group, a substituted or unsubstituted alkyl group, a substituted or unsubstituted aryl group, a substituted or unsubstituted aralkyl group, a substituted or unsubstituted heterocyclic group.

3. The compound as claimed in claim 1, wherein the biphenyl group is represented by the general formula (II): 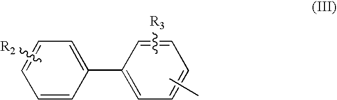
wherein R₂ and R₃ are selected from a hydrogen atom, a halogen atom, a substituted or unsubstituted amino group, a substituted or unsubstituted alkyl group, a substituted or unsubstituted aryl group, a substituted or unsubstituted aralkyl group, a substituted or unsubstituted heterocyclic group.

4. The compound as claimed in claim 1, wherein the fluorenyl group is represented by the general formula (II): 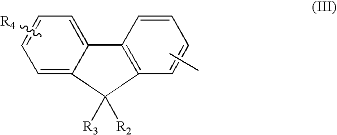
wherein R₄ is selected from a hydrogen atom, a halogen atom, a substituted or unsubstituted amino group, a substituted or unsubstituted alkyl group, a substituted or unsubstituted aryl group, a substituted or unsubstituted aralkyl group, a substituted or unsubstituted heterocyclic group and R₅ and R₆ are selected from a substituted or unsubstituted alkyl group, a substituted or unsubstituted aryl group, a substituted or unsubstituted aralkyl group

5. The compound as claimed in claim 1, wherein the anthryl group is represented by the general formula (II): 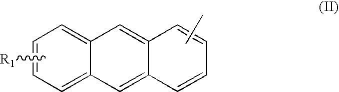
wherein R₁ is selected from a hydrogen atom, a halogen atom, a substituted or unsubstituted amino group, a substituted or unsubstituted alkyl group, a substituted or unsubstituted aryl group, a substituted or unsubstituted aralkyl group, a substituted or unsubstituted heterocyclic group.

6. A organic light emitting device comprising a pair of electrodes consisting of a cathode and an anode and between the pairs of electrodes comprising a layer of anthracene compound represented as the following formula [I] 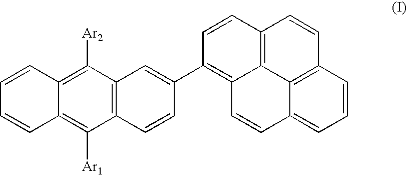
Wherein Ar₁ and Ar₂ are the same or different and each is selected from a substituted or unsubstituted phenyl group, a substituted or unsubstituted naphthyl group, a substituted or unsubstituted biphenyl group, a substituted or unsubstituted fluorenyl group, a substituted or unsubstituted anthryl group.

7. According to claim 6, an organic light emitting device comprising a layer of a anthracene compound represented as formula [I] and functions as emitting host of a light emitting layer.
